(19) United States
(12) Patent Application Publication
(10) Pub. No.: US 2003/0127090 A1
(43) Pub. Date: Jul. 10, 2003

(54) Active pump bronchial implant devices and methods of use thereof

(75) Inventors: Hanson S. Gifford, Woodside, CA (US); John G. McCutcheon, Menlo Park, CA (US); Antony J. Fields, San Francisco, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(73) Assignee: Emphasys Medical, Inc.

(21) Appl. No.: 10/298,387

(22) Filed: Nov. 14, 2002

Related U.S. Application Data
(60) Provisional application No. 60/336,233, filed on Nov. 14, 2001.

(51) Int. Cl. A61F 2/04

(52) U.S. Cl. 128/200.24; 623/9; 623/23.65; 600/16

(57) ABSTRACT

Disclosed is a pump device that can be implanted into a body passageway, such as into a bronchial passageway. The pump device can be used to pump fluid through the body passageway, such as in order to assist the expiration of fluid from a region of the lung that fluidly communicates with the body passageway. The pump device includes a housing that defines an internal chamber, wherein fluid can flow through the chamber. The housing is dimensioned for insertion into a bronchial passageway. The pump device also includes a fluid propulsion mechanism in fluid communication with the chamber. The fluid propulsion mechanism is positioned to propel fluid through the chamber so as to pump fluid through the bronchial passageway in a desired direction.

### REFERENCE TO PRIORITY DOCUMENT

This application claims priority of co-pending U.S. Provisional Patent Application Serial No. 60/336,233 entitled “Active Pump Bronchial Implant Devices” by H. Gifford et al., filed Nov. 14, 2001. Priority of the filing date of Nov. 14, 2001 is hereby claimed, and the disclosure of the Provisional Patent Application is hereby incorporated by reference.

### BACKGROUND OF THE INVENTION

1. Field of the Invention

This invention relates generally to methods and devices for use in performing pulmonary procedures and, more particularly, to procedures and devices for treating various diseases of the lung.

2. Description of the Related Art

Emphysema is a condition of the lung characterized by the abnormal permanent enlargement of the airspaces distal to the terminal bronchiole, accompanied by the destruction of their walls, and without obvious fibrosis. (Snider, G. L. et al: The Definition of Emphysema: Report of the National Heart Lung And Blood Institute, Division of lung Diseases Workshop. (Am Rev. Respir. Dis. 132:182, 1985)).

It is known that emphysema and other pulmonary diseases reduce the ability of one or both lungs to fully expel air during the exhalation phase of the breathing cycle. The diseased lung tissue is less elastic than healthy lung tissue, which is one factor that prevents full exhalation of air and can also contribute to hyperexpansion of the lung. During breathing, the diseased portion of the lung does not fully recoil, due to the tissue being less elastic. Consequently, the diseased lung tissue exerts a relatively low driving force, which results in the diseased lung expelling less air volume than a healthy lung. The reduced air volume exerts less force on the airway, which allows the airway to close before all air has been expelled, another factor that prevents full exhalation.

In addition, hyper-expanded lung tissue occupies more of the pleural space than healthy lung tissue. In most cases, a portion of the lung is diseased while the remaining part is healthy and, therefore, still able to efficiently carry out oxygen exchange. By taking up more of the pleural space, the hyper-expanded lung tissue reduces the amount of space available to accommodate the healthy, functioning lung tissue. As a result, the hyper-expanded lung tissue causes inefficient breathing due to its own reduced functionality and because it adversely affects the functionality of adjacent healthy tissue.

Lung reduction surgery is a conventional method of treating lung diseases such as emphysema. A diseased portion of the lung is surgically removed, which makes more of the pleural space available to accommodate the functioning, healthy portions of the lung. The lung is typically accessed through a median sternotomy or small lateral thoracotomy. A portion of the lung, typically the upper lobe of each lung, is freed from the chest wall and then resected, e.g., by a stapler lined with bovine pericardium to reinforce the lung tissue adjacent the cut line and also to prevent air or blood leakage. The chest is then closed and tubes are inserted to remove air and fluid from the pleural cavity. The conventional surgical approach is relatively traumatic and invasive, and, like most surgical procedures, is not a viable option for all patients.

What has been needed are improved methods and devices for performing pulmonary procedures, such as the removal of air or fluid from a portion of the lung.

### SUMMARY

Disclosed is a pump device that can be implanted into a body passageway, such as into a bronchial passageway. The pump device can be used to pump fluid through the body passageway, such as in order to assist the expiration of fluid from a region of the lung that fluidly communicates with the body passageway. The pump device includes a housing that defines an internal chamber, wherein fluid can flow through the chamber. The housing is dimensioned for insertion into a bronchial passageway. The pump device also includes a fluid propulsion mechanism in fluid communication with the chamber. The fluid propulsion mechanism is positioned to propel fluid through the chamber so as to pump fluid through the bronchial passageway in a desired direction.

Also disclosed is a method of assisting expiration from a patient's lung, comprising implanting a pump into a bronchial lumen that fluidly communicates with the lung and operating the pump so that the pump causes gas to flow out of the patient's lung through the bronchial lumen while the pump is positioned within the bronchial lumen.

Embodiments for methods of using the pumping devices provide for the removal of fluid within an intracorporeal lumen or lung segment that can include providing an intracorporeal pump device having features described above or a combination thereof and advancing the intracorporeal pump through a patient's pulmonary system. The method further includes placing the pump device within a bronchial lumen such that the pump device seals to the bronchial lumen. The pump device is then actuated to effect a unidirectional movement of fluid flow through the device in an expiratory direction from an internal segment of the lung.

These and other features, aspects and advantages of embodiments of the present invention will become better understood with regard to the following description, appended claims, and accompanying drawings.

### BRIEF DESCRIPTION OF THE DRAWINGS

FIG. 1A is a side view in partial cross-section of a pump device implanted in a bronchial passageway.

FIG. 1B is an end view of the pump device of FIG. 1A.

FIG. 2 is a schematic view of the pump device placed within the right main upper lobe bronchus of a patient.

FIG. 3A is a longitudinal cross-sectional view of an embodiment of a thermally activated pump device disposed within an intracorporeal lumen.

FIG. 3B is a cross-sectional view of the pump device of FIG. 3A in an expanded state.

FIG. 4A is an end view of the pump device of FIG. 3A.

FIG. 4B is a transverse cross-sectional view of the device of FIG. 3B taken along lines 6-6 of FIG. 3B.

FIG. 5 is a top view of the pump device of FIG. 3B taken along line A of FIG. 3B.

FIG. 6 is a side view of the pump device of FIG. 3B taken along line B of FIG. 4A.

FIG. 7 is a longitudinal cross-sectional view of the pump device of FIG. 3A showing the pump device in a contracted state in response to a temperature change.

FIG. 8 is a longitudinal cross-sectional view of an embodiment of a magnetically-activated pump device disposed within an intracorporeal lumen.

FIG. 9 is a transverse cross-sectional view of the pump device of FIG. 8 taken along line 9-9 of FIG. 8.

FIG. 10 is an expanded view of the magnetic pump device of FIG. 8.

FIG. 11 is an expanded view of the magnetically-activated pump device of FIG. 10 taken along circle 11 of FIG. 10.

FIG. 12 is an expanded view of the magnetically-activated pump device of FIG. 10 taken along circle 11 showing lateral motion of the actuation member.

FIG. 13 is a longitudinal cross-sectional view of the pump device of FIG. 8 shown in a contracted state in response to an external magnet placed along the chest wall.

FIG. 14 is a transverse cross sectional view of the pump device of FIG. 13 taken along lines 14-14 of FIG. 13.

FIG. 15 is a longitudinal cross-sectional view of an embodiment of a magnetically driven pump device shown in a contracted state.

FIG. 16 is a transverse cross sectional view of the pump device of FIG. 15 taken along lines 16-16 of FIG. 15.

FIG. 17 is a longitudinal cross-sectional view of the magnetically driven pump device shown in FIG. 15 in a retracted state in response to an external magnet placed along the chest wall.

FIG. 18 is a transverse cross sectional view of the device of FIG. 17 taken along lines 18-18 of FIG. 17.

FIG. 19 is a longitudinal cross-sectional view of another embodiment of a magnetically driven pump device.

FIG. 20 is a longitudinal cross-sectional view of the magnetically driven pump device of FIG. 19 in a compressed state in response to an external magnet placed along the chest wall.

FIG. 21 is a longitudinal cross-sectional view of the magnetically driven pump device of FIG. 19 in a further compressed state in response to the external magnetic source placed along the chest wall.

FIG. 22 shows a longitudinal cross-sectional view of a fixed-volume chamber pump device placed within a bronchial lumen.

FIG. 23 shows a transverse cross sectional view of the pump device of FIG. 22, taken along lines 23-23 of FIG. 22.

FIG. 24 shows a schematic view of another embodiment of a fixed volume pump device placed within the right main upper lobe bronchus of a patient.

FIG. 25 shows a longitudinal cross-sectional view of an embodiment of a moveable weight pump device.

FIG. 26 shows a bronchoscope deployed within a bronchial tree of a patient.

### DETAILED DESCRIPTION

Embodiments of methods and pump devices for use in performing pulmonary procedures and more particularly for treating various lung diseases, such as emphysema, are described herein. Embodiments and uses thereof provide for a unidirectional flow of fluid through a chamber implanted in a bronchial lumen; such as to effect fluid flow in an exhalation direction in relation to the bronchial lumen and prevent fluid flow through a chamber in an inhalation direction. As used herein the term fluid means gas, liquid or a combination of gas(es) and liquid(s). The pump device can be implanted in a bronchial lumen and used to pump fluid into or out of a region of a lung, such as an isolated lung region.

Pump Device

Disclosed is a pump device that can be implanted into a body passageway, such as into a bronchial passageway. The pump device can be used to pump fluid through the body passageway, such as in order to assist the expiration of fluid from a region of the lung that fluidly communicates with the body passageway.

In one embodiment, the pump device includes a housing that defines an internal chamber, wherein fluid can flow through the chamber. The housing is dimensioned for insertion into a bronchial passageway. The pump device also includes a fluid propulsion mechanism in fluid communication with the chamber. The fluid propulsion mechanism is positioned to propel fluid through the chamber so as to pump fluid through the bronchial passageway in a desired direction. Embodiments of the pump device with various embodiments of the housing and propulsion mechanism are described below.

The pump device further includes a retainer that can be used to retain the pump device in a fixed location within the bronchial lumen. When the pump device is implanted in a bronchial passageway, the retainer exerts a force against the bronchial wall of the passageway. The force is sufficient to retain the pump device in a fixed position relative to the bronchial wall. The pump device can also include a sealing member that provides a seal between the pump device and the bronchial wall in which the pump device is implanted, so that fluid in the bronchial passageway must flow through the internal chamber in order to flow across the pump device.

The propulsion mechanism can be coupled to a drive mechanism that causes the propulsion mechanism pump fluid. As described in detail below, the drive mechanism can utilize various mechanisms to impart motion to the propulsion mechanism, such as magnets or shape-memory and temperature-sensitive materials. The pump device can be coupled to a power supply that provides power to the propulsion mechanism. Power can be obtained in a variety of manners, such as by using an electrical battery, or by converting mechanical movement into energy. For example, the movement of the patient's body can be utilized to impart motion to the drive mechanism and propulsion mechanism. The potential energy of gravity can also be utilized to power the propulsion mechanism.

The pump device can optionally be coupled to at least one valve that fluidly communicates with the internal chamber. The flow of fluid through the chamber is controlled by the valve, which is disposed at a location along the chamber such that fluid must flow through the valve in order to flow through the chamber, as described more fully below. As described below, the valve can be a one-way valve that permits fluid to flow through the chamber only in one direction.

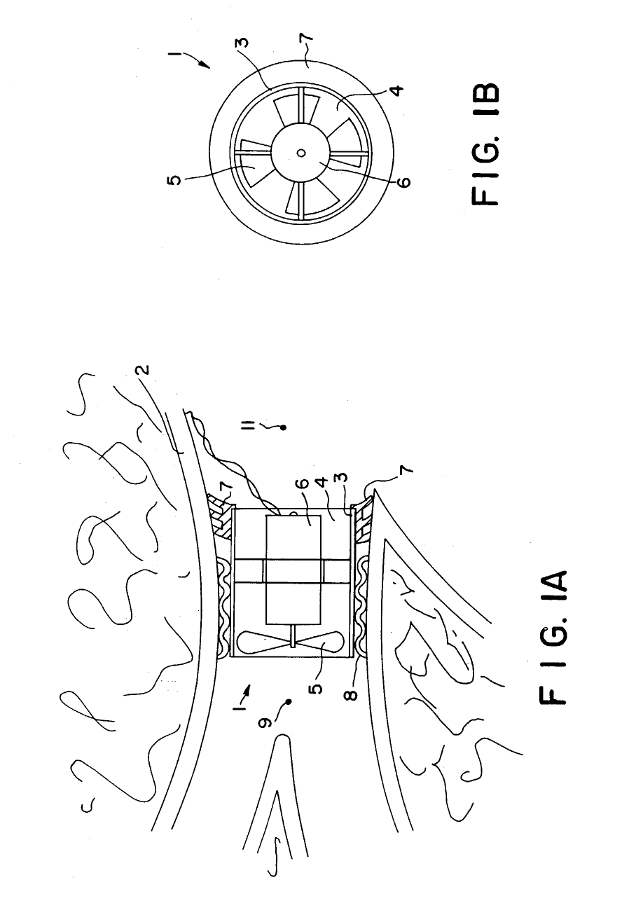

FIG. 1A shows one embodiment of a pump device 1 mounted within a body passageway, such as a bronchial passageway 2. FIG. 1B shows an end view of the pump device 1. The pump device includes an annular housing 3 that forms an interior chamber 4 through which fluid can flow. The pump device 1 further includes a propulsion mechanism comprised of a fan or impeller 5. The impeller 5 is coupled to a drive mechanism comprised of a motor 6 that is mounted within the interior chamber 4. A sealing member 7 is disposed on an outer surface of the housing 3 for sealing to an interior wall of the bronchial passageway 2. The pump device 1 further includes a retainer 8 that retains the pump device 1 in a fixed position within the bronchial passageway 2.

With reference still to FIGS. 1A and 1B, the motor 6 is suspended inside the interior chamber 4 such that there is an open annulus of space between the outer diameter of the motor and the inner diameter of the housing 3. This allows fluid to flow through the interior chamber around the motor 6. In one embodiment, the motor 6 is a brushless, electromagnetic, direct-current motor. The impeller 5 is mechanically coupled to the motor (such as through a drive shaft) so that the motor can drive the impeller 5 to cause the impeller to spin and effect a fluid flow through the interior chamber 4 and through the bronchial passageway 2. Thus, as the impeller rotates, fluid is drawn into the chamber from a distal side 9 of the pump device 1, through the blades of the impeller 5, and around the motor 6. The fluid is then expelled from the interior chamber 4 to a proximal side 11 of the pump device 1. The impeller 5 can be mounted at any location on the pump device 1 that will enable the impeller to effect a fluid flow through the pump device 1.

The retainer 5 can comprise, for example, an expandable frame or stent that is mounted on an external surface of the housing 3. The retainer 5 can be mounted at any location on the pump device that will enable the retainer 5 to exert a force against the walls of the bronchial passageway. The retainer 5 can also be mounted distal or proximal to the housing 3. In another embodiment, the pump device is retained in place by an inflatable balloon that is mounted to the outside diameter of the housing 3.

With reference to FIGS. 1A and 1B, the sealing member 7 is located on the outside surface of the housing 3. The seal member 7 can have any of a wide variety of shapes that will provide a seal between the outer surface of the housing 3 and the interior wall of the bronchial passageway 2. For example, in the illustrated embodiment, the seal member 7 includes a plurality of flanges that extend radially-outward from the pump device 1 and contact the bronchial passageway.

The sealing member 7 and/or the retainer 5 can contract or expand in size, particularly in a radial direction. The default state is preferably an expanded size, such that the pump device will have a maximum diameter (which is defined by either the seal or the retainer) when the pump device is in the default state. Thus, the pump device can be radially contracted in size during insertion into a bronchial passageway, so that once the pump device is inserted into the passageway, it expands within the passageway. The size expansion/contraction characteristics can be enabled using the retainer, such that the retainer can be self-expanding. Thus, the retainer can be in at least two states, including an insertion (compressed) state and an anchoring (expanded) state. In the insertion state, the retainer has a smaller diameter than in the anchoring state. Various mechanisms can be employed to achieve the two states. In one embodiment, the retainer is manufactured of a malleable material. The retainer can be manually expanded to the anchoring state, such as by inserting an inflatable balloon inside the retainer once the pump device is implanted in the bronchial passageway, and then inflating the balloon to expand the retainer beyond the material's yield point into an interfering engagement with the wall of the bronchial passageway.

Another mechanism that can be employed to achieve the two-state retainer size is spring resilience. The insertion state can be achieved through a preconstraint of the retainer within the elastic range of the retainer material. Once positioned in the bronchial passageway, the retainer can be released to expand into an anchoring state. Constraining tubes or pull wires may achieve the initial insertion state.

Another mechanism that can be used to achieve both the insertion and the anchor states of the retainer is the heat recovery of materials available with alloys, such as certain nickel titanium alloys, including Nitinol. The transition temperature of the retainer could be below body temperature. Under such a circumstance, a cool retainer can be positioned and allowed to attain ambient temperature. The unrecovered state of the retainer would be in an insertion position with the retainer having a smaller diameter. Upon recovery of the retainer material, the retainer would expand, such as when the retainer achieves a temperature within the bronchial passageway. Another use of this material may be through a heating of the device above body temperature with a recovery temperature zone above that of normal body temperature but below a temperature which may cause burning. The device might be heated electrically or through the modulation of a field.

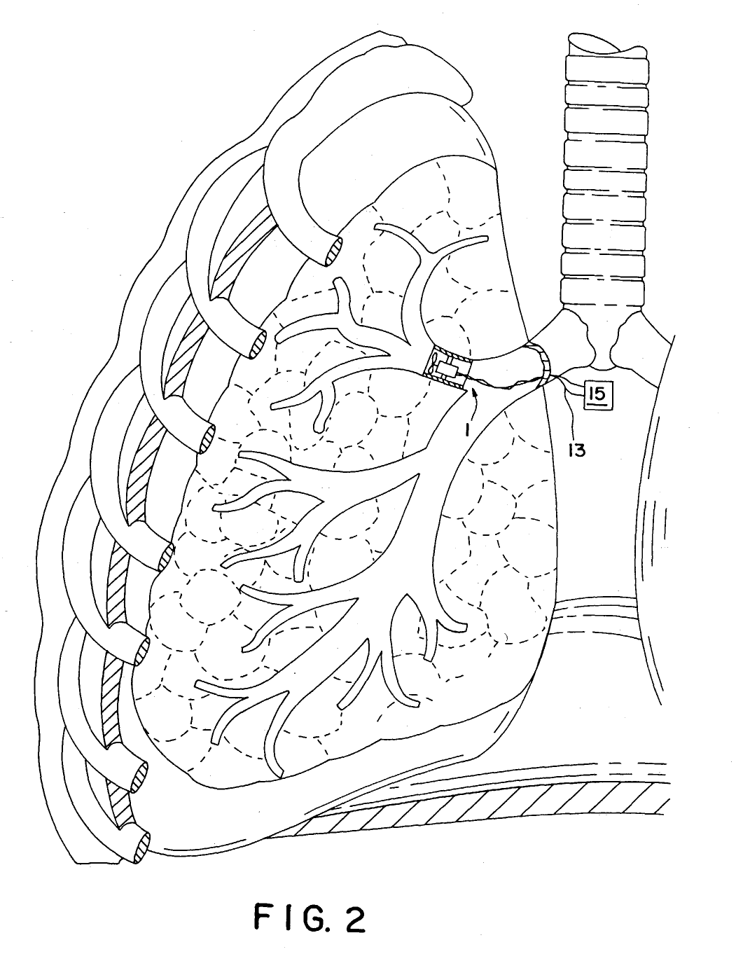

FIG. 2 shows the pump device 1 mounted within the right main upper lobe bronchus of a patient. As mentioned, in one embodiment, the power supply comprises a battery that provides electrical power to the pump device. FIG. 2 schematically shows a battery 15 located outside of the pump device. The battery can be located within the pump device, or it can be located outside of the pump device. If the battery is located outside of the pump device, the battery can be located either inside or outside of the patient's body. When inside the body, the battery can be located within the lungs, or it can be located outside of the lungs (i.e., subcutaneously located), and connected to the pump device via wires 13 (shown in FIG. 2) that run through the bronchial wall at a desired location. The battery can be charged either directly with a plug through the patient's skin, or the battery can preferably be charged inductively. Alternately, the battery can be located outside of the patient's body using wires that percutaneously communicate with the pump device.

The pump device can pump fluid either intermittently or continuously. For example, the pumping of fluid can be timed to coincide with the patient's breathing cycle, such as in synchronization with the patient's inhalation, exhalation, or both. This can be accomplished, for example, by alternately turning an attached power source, such as a battery, on and off in synchronization with the timing of the breathing. The timing of the breathing cycle can be determined by sensing body indicators associated with the breathing cycle (e.g., sensing nerve impulses, sensing expiration chest movement, sensing bronchial wall movement, sensing gas concentration in lung, etc.) The pumping action can also be made to coincide with certain time intervals, rather than with the breathing cycle.

Thermally-Activated Pump Device

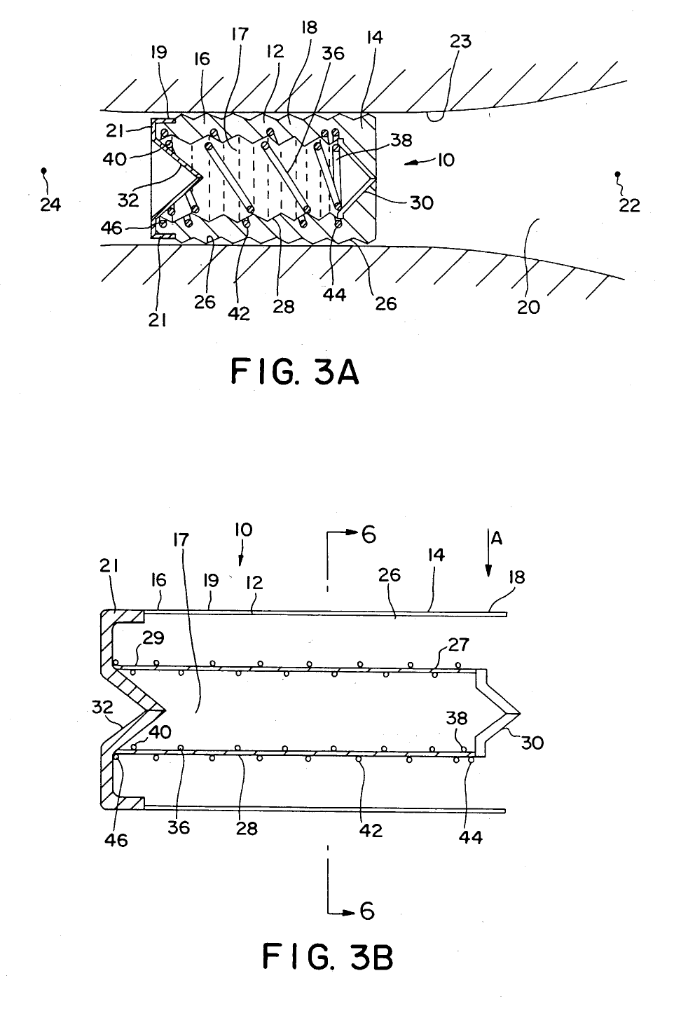

FIGS. 3-7 illustrate another embodiment of a pump device 10 that provides fluid pumping within an intracorporeal lumen, such as a bronchial lumen. The pump device has an internal chamber actuator that is responsive to changes in temperature such that the actuator alters the volume of the chamber in response to changes in temperature. The change in volume effects a pumping action that can be used to pump fluid through the bronchial lumen in a desired flow direction, such as in an expiratory direction, in order to expel fluid from a region of the lung. In one embodiment, the actuator comprises a pair of springs that collectively define the volume of the chamber. The springs are each manufactured of a temperature-sensitive material that has shape-memory properties.

FIGS. 3A and 3B show a longitudinal cross-sectional view of an embodiment of the thermally activated pump device 10. The pump device 10 has a body portion 12 with a proximal (i.e., closer to the trachea) section 14 and a distal (i.e., closer to the lung segment) section 16. The body portion 12 forms a chamber 17 that is disposed internally within the body portion 12 and surrounded circumferentially by the body portion 12 along a longitudinal length between the proximal section 14 and the distal section 16 of the body portion 12. The body portion 12 is formed of a tubular member 18 made of a compliant material, such as Polytetrafluoroethylene (PTFE), but can also be made of silicon or another biocompatible polymer. The tubular member 18 as shown in FIG. 3A is positioned within an intracorporeal lumen comprised of a bronchial lumen 20 with the proximal section 14 adjacent to a proximal side 22 of the bronchial lumen and with the distal section 16 adjacent to a distal side 24 of the bronchial lumen 20. In an exemplary embodiment, the tubular member 18 has an axial length of about 10 millimeters (mm) to 30 mm, although the length could also be outside this range. The tubular member has an external diameter that would permit the member to disposed within various bronchial passageways in the bronchial tree, or other intracorporeal lumens. In one embodiment, the outer diameter of the tubular member is about 5 mm to about 15 mm, although the diameter can vary based on the size of the bronchial passageway.

The tubular member 18 has a sealing member 21 that is located on an external surface 19 of the body portion 12. The sealing member 21 forms a seal with an internal surface of an intracorporeal lumen such that the sealing member 21, as shown in FIG. 3A, forms a seal along an external surface 19 of the tubular member 18 to an internal surface 23 of the bronchial lumen 20. When the tubular member is disposed within the bronchial lumen 20, the seal prevents the flow of fluid around the device 10 within the lumen 20 such that fluid flow is prevented between the external surface 19 of the tubular member 18 and the internal surface 23 of the bronchial lumen 20. The sealing member 21 can be made of a soft material, such as a polymer, including, for example, silicone, having a durometer, for example, of about 5 Shore A to about 90 Shore A, but can also be made of other biocompatible materials having various durometer values. The sealing member 21 can have an outer dimension that substantially matches the inner dimension of the bronchial lumen 20 so that the sealing member 21 fits snugly with the bronchial lumen 20.

The pump device 10 can include a retainer that functions to retain the pump device 10 in a fixed position within the bronchial lumen 20. For example, the pump device 10 can include a self-expanding retainer 26 that is circumferentially disposed within the body portion 12 of the device 10. The self-expanding retainer 26 imparts a radially directed outward force to secure the body portion 12 of the device 10 against the internal surface 23 of the bronchial lumen 20. The self-expanding retainer 26 is made of a suitable biocompatible material that can expand in size when implanted in a lumen. In one embodiment, the self-expanding retainer 26 is laser cut from a Nitinol tube, but it should be appreciated that the self-expanding retainer 26 can also be made from other expandable materials, such as stainless steel, or the like. Various types of sealing members and retainers can be used, such as the sealing members and retainers described in the U.S. patent application Ser. No. 10/270,792, entitled “Bronchial Flow Control Devices and Methods of Use”, which is assigned to the same assignee as the instant application and which is incorporated herein by reference in its entirety.

In one embodiment, the retainer comprises a frame formed by a plurality of struts that define an interior envelope so that the frame can be sized to surround the implantable pump device. The struts of the frame can form curved, proximal ends that can be slightly flared outward with respect to a longitudinal axis of the pump device. When the pump device is placed in a bronchial lumen, the curved, proximal ends can anchor into the bronchial walls and prevent migration of the pump device in a proximal direction. The frame can also have flared, distal prongs that can anchor into the bronchial walls and to prevent the pump device from migrating in a distal direction when the flow pump device is placed in a bronchial lumen. The frame can be formed from a super-elastic material, such as Nitinol, such as by cutting the frame out of a tube of Nitinol or by forming the frame out of Nitinol wire. The super-elastic properties of Nitinol can result in the frame exerting a radial force against the interior walls of a bronchial passageway sufficient to anchor the pump device in place. The struts can be arranged so that the frame can expand and contract in a manner that is entirely or substantially independent of the rest of the pump device. It should be appreciated that the frame does not necessarily have to be built in this manner and that the frame can have other configurations.

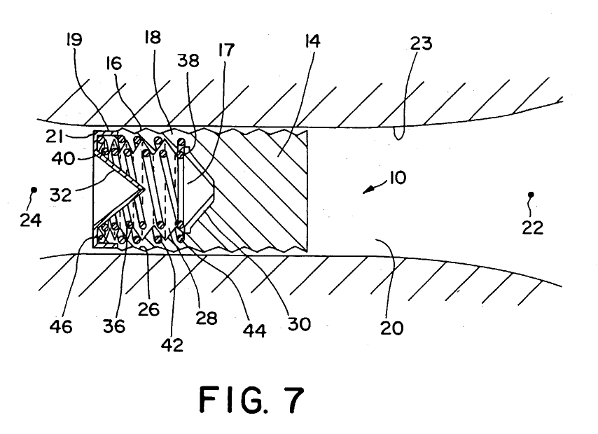

As best shown in FIGS. 3A and 3B, disposed within the tubular member 18 is a fluid propulsion mechanism comprised of a bellows 28 that is disposed circumferentially around the chamber 17. The bellows 28 is configured to expand and contract between two different states, including an expanded state (as shown in FIGS. 3A and 3B) wherein the chamber 17 has a first volume, and a contracted state (as shown in FIG. 7) wherein the chamber 17 has a second volume that is smaller than the first volume, as described more fully below.

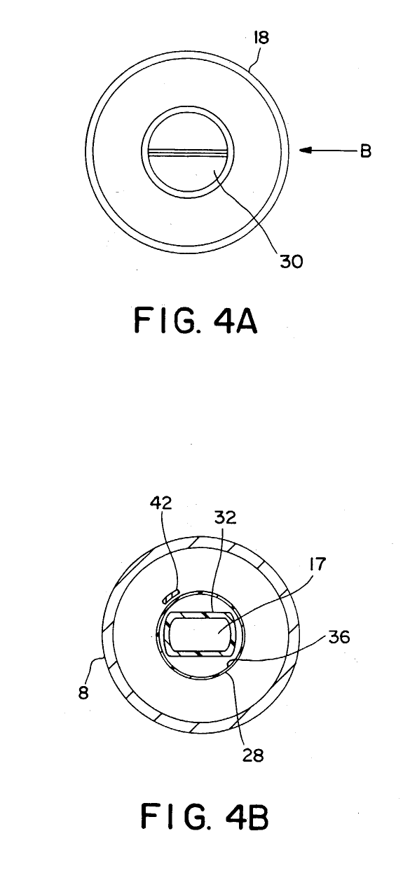

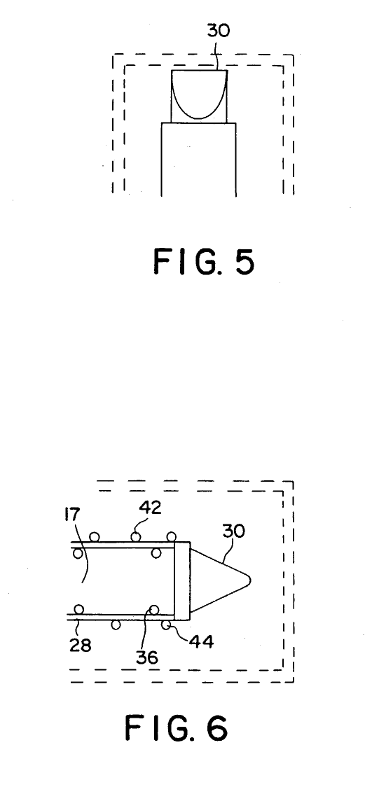

A proximal one-way valve 30 is attached and sealed to a proximal side 27 of the bellows 28 and a distal one-way valve 32 is attached and sealed to a distal side 29 of the bellows 28. The valves 30, 32 can be open, wherein the valves permit fluid to flow therethrough, or the valves 30, 32 can be closed, wherein the valves do not permit fluid to flow therethrough. FIG. 4A shows an end view of the pump device 10 and the proximal valve 30 in a closed position. FIG. 5 is a top view and FIG. 6 is a partial side view of the pump device.

The proximal one-way valve 30 and the distal one-way valve 32 can be positioned within the pump device 10 to cooperatively allow the unidirectional flow of fluid through the chamber 17 in a desired direction, such as an expiratory direction (toward the trachea). The valves 30, 32 can comprise, for example, duck bill valves that permit fluid to flow in a first direction (such as an expiratory direction) but prohibit fluid from flowing in a second direction (such as an inhalation direction) that is opposed to the first direction. Other types of valves can be used, such as the valves described in the aforementioned U.S. patent application entitled “Bronchial Flow Control Devices and Methods of Use”, as wells as the valves described in U.S. Pat. No. 5,954,766, entitled “Body Fluid Flow Control Device”, which is assigned to the same assignee as the instant application and which is incorporated herein by reference in its entirety.

The valves 30, 32 can be made of a soft material, such as a soft polymer including silicone having a durometer, for example, of about 5 Shore A to about 90 Shore A, but can also be made of other biocompatible materials having various durometer values. The valves 30, 32 can also be made from any other biocompatible polymer having a suitable durometer. The valves have an outer dimension that permits the valve to fit within the tubular member 18. FIG. 4B shows a cross-section of the device of FIG. 3B taken along line 6-6 of FIG. 3A and shows the distal one-way valve 32 disposed within the bellows 28 and sealed to the sealing member 21. It should be appreciated that the valves 30, 32 are not limited to duckbill configurations. For example, in other embodiments the valves can have a configuration such as poppet, ball, leaflet, Heimlich, reed, diaphragm, and flap valves or the like. Each of the proximal and distal one-way valves may have the same configuration, or a combination of different valve configurations and placements can be used.

As mentioned, the bellows 28 is configured such that it is axially collapsible along its longitudinal length into a contracted state. More particularly, the bellows 28 is positioned such that it can contract toward the distal section 16 (as shown in FIG. 7) into a contracted state. The bellows can also expand toward the proximal section 14 into an expanded state (as shown in FIGS. 3A and 3B), which can be its default state. The bellows 28 transitions between the contracted and expanded state based on the temperature of the environment of the pump device 10, as described below.

The bellows is made of a material that can expand and contract, such as, for example silicone, but can be made of other materials such as polyurethane or the like. In one embodiment, the bellows is made of a cloth material, such as Dacron, or the bellows is reinforced with fibers, in order to extend the fatigue life of the bellows. Alternately, the bellows may be constructed of an elastomer, such as polyurethane, that is reinforced with a cloth-like material such as Dacron.

Mounted circumferentially within the bellows 28 between the proximal one-way valve 30 and the distal one-way valve 32 is a drive mechanism, such as an elastic coil spring 36, that causes the bellows to expand and contract. The elastic coil spring 36 is attached at a proximal end 38 to the proximal one-way valve 30 and attached at a distal end 40 to the distal one-way valve 32. The elastic coil spring 36 separates and sets the proximal one-way valve 30 and the distal one-way valve 32 apart at desired relative position when the bellows is at the expanded state as shown in FIG. 3A. That is, the coil spring 36 maintains the bellows 28 in an expanded state by exerting a spring force that, unless overcome by a stronger force, will maintain the bellows 28 in the expanded state. The elastic coil spring is made of a material that can withstand alternate contraction and expansion, such as steel, but can also be made of Nitinol, or the like.

Positioned circumferentially about the bellows 26 is a shape-memory coil spring 42 that is disposed between the proximal one-way valve 30 and the distal one-way valve 32. The shape-memory coil spring 42 is attached at a proximal end 44 and a distal end 46 to the proximal one-way valve 30 and distal one-way valve 32, respectively. The shape-memory coil spring 42 is made of a temperature-sensitive material that has properties that vary with temperature. For example, the shape-memory coil spring can be made of a temperature-sensitive material, such as Nitinol, that has a predetermined transition temperature at which the properties of the material change. Below the transition temperature, the spring force of the shape-memory coil spring 42 is less than above the actuation temperature. In one embodiment, the transition temperature of the shape-memory coil spring 42 is normal body temperature (37° C.), such that the properties of the spring 42 are different below body temperature than above normal body temperature.

The shape-memory coil spring 42 exerts a force that opposes the force of the elastic coil spring 36. Below the transition temperature, the spring force exerted by the shape-memory coil spring 42 is insufficient to overcome the force exerted by the elastic coil spring 36, so that the bellows is maintained at the expanded state. Above the transition temperature, the force exerted by the shape-memory coil spring 42 overcomes the force exerted by the elastic coil spring 36, so that the shape-memory coil spring 42 retracts and causes the bellows to move to the contracted state.

Thus, at a certain temperature, the proximal one-way valve 30 and distal one-way valve 32 are separated and maintained in the expanded state as shown in FIG. 3A by the elastic coil spring 36. When the device 10 is heated, such as by warm air, the shape-memory coil spring 42 contracts to overcome the tension of the elastic coil spring 36 and hence, draw the proximal one-way valve 30 and the distal one-way 32 valve together as shown in FIG. 7. The contraction of the spring reduces the volume of the chamber 17 to thereby expel fluid out of the chamber 17 through the proximal valve 30. The change in the chamber volume effects a pressure change between the chamber 17 and the proximal one-way valve 30 and the proximal side of the lumen 22. The volume change also effects a pressure change between the distal one-way valve 32 and the distal side of the lumen 24. The pressure changes effect fluid flow through the chamber 17.

The shape-memory coil spring 42 can be made of a biocompatible, temperature-sensitive material that has shape-memory, such as Nitinol. In further embodiments, heating or cooling of the shape-memory coil spring 42 can be induced by means of heating or cooling elements associated with the pump device. For example, the shape-memory coil spring 42 may be connected to an electrical power source, such as a battery that delivers power to the shape-memory coil spring 42, thereby causing it to heat up. The battery may be mounted within the pump device or externally mounted within or outside the patient's chest.

The proximal one-way valve 30 and the distal one-way valve 32 have various configurations and combinations thereof and can have various cracking pressures, such as, for example, a cracking pressure of about 0.005 psi to about 0.4 psi, such that the proximal one-way valve 30 and distal one-way valve 32 are sensitive to slight volume changes within the chamber 17. In one embodiment, a lower cracking pressure is more desirable. As mentioned, the volume changes within the chamber 17 effect a pressure change between the chamber 17 and the proximal side 22 of the bronchial lumen 20 and the chamber 17 and a distal side 24 of the bronchial lumen 20. The pressure change allows for fluid to be pumped into the chamber from a portion of the lung via a distal side 24 of the bronchial lumen 20 through the distal one-way valve 32. Fluid can also be pumped out of the chamber 17 through the proximal one-way valve 30. The fluid can then be removed from the body.

A volume change due to the activation of the shape-memory coil spring 42 can induce a pressure change between the chamber 17 and the proximal side of the lumen 22 and the chamber 17 and the distal side of the lumen 24. The resulting pressure differentials can induce an opening within either the proximal one way valve 30 and the distal one-way valve 32, which can induce fluid flow through the chamber 17.

In additional embodiments, the actuation member may be sealed within the body of the pump device or have a configuration which allows for the pump to function in the presence of mucous. An additional embodiment could include a pump configured with a pin device or a small hole disposed within the chamber 17 at either end. The pin could pierce the small hole at the desired end to clear the chamber of mucous or other more viscous fluid and prevent the chamber from clogging over.

The shape-memory coil spring 42 can have a large surface area to mass ratio such that it can be heated or cooled very quickly to expand or retract in response to temperature changes within the bronchial lumen 20. These temperature changes can be effected when the device 10 is exposed to fluid flow within the bronchial lumen 20 which can be created when the patient breathes air of various temperatures. A cyclic pumping action can be effected by the patient upon intermittent breathing of cold or warm air. In other embodiments the temperature changes can be created by normal respirations of air at room temperature or other various environmental temperatures. A cyclic pumping action can be effected by the patient upon intermittent breathing of cold or warm air. Typically the actuation member can be cooled and heated rapidly as described and is responsive to minor temperature changes such as those that occur with normal respirations or those that may be imparted within the lumen by the patient breathing the very warm or very cold air. The pumping action is preferably synchronized with the patient's breathing cycle, such that the pump device 10 pumps in synchronization with the patient's breaths. This can be accomplished, for example, by turning an attached power source, such as a battery, on and off in synchronization with the breathing, such as by using a timer or by sensing body indicators associated with the breathing cycle (e.g., sensing nerve impulses, sensing expiration chest movement, sensing the percentage of certain gas concentrations in lung, etc.).

Alternate embodiments of thermally activated shape-memory driven pumps can include an actuation member such as a shape-memory ring, coil, stent-like structure or the like having various configurations and placements within the pump device. Alternate embodiments of the present device include intracorporeal pump devices that have actuation members which are sealed within the body portion and those which are configured to work in the presence of mucous. It should be appreciated that other mechanisms can be used to alter the volume of the chamber. For example, a plunger can be movably located within the chamber. The plunger can move back and forth within the chamber so that the plunger consumes varying amounts of volume within the chamber to thereby cyclically change the volume of the chamber. The actuation member can also be driven by means other than the temperature-sensitive characteristics of a spring, such as by using magnets in combination with magnetic forces, using the patient's body movements to impart power to the actuation member, or using potential energy associated with gravity.

Magnetically-Actuated Pump Device

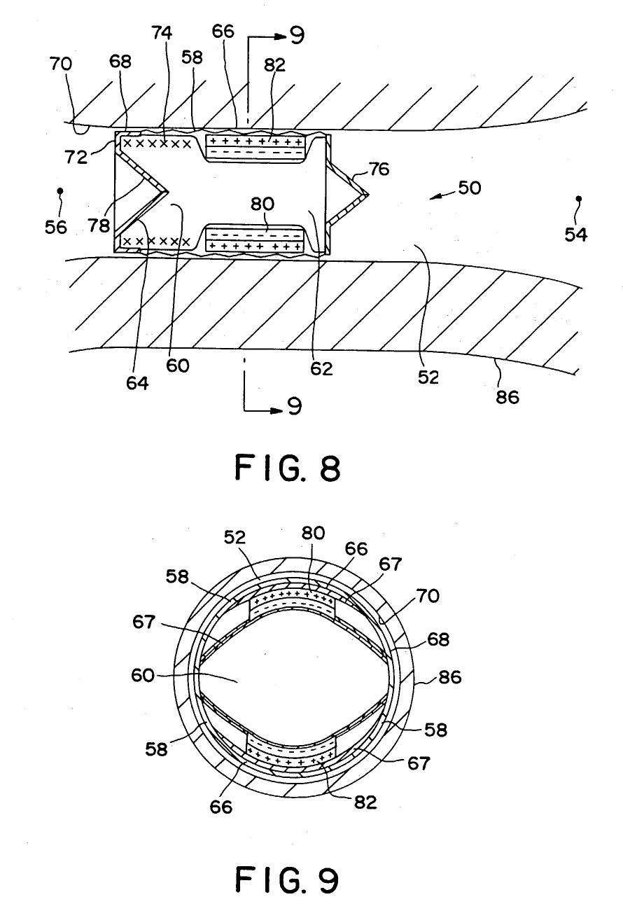

FIGS. 8-14 show a magnetically actuated pump device 50. FIG. 8 shows a longitudinal cross sectional view of the pump device 50 and FIG. 9 shows a transverse cross-sectional view of the pump device 50. The pump device 50 is disposed within a bronchial lumen 52 and positioned such that the pump device 50 will move fluid (gas or liquid) from the distal side 56 of the bronchial lumen 52 to the proximal side 54 of the bronchial lumen 52. The device 50 has a body portion 58 with a chamber 60 disposed internally within the body portion 58. A proximal section 62 of the device 50 is positioned adjacent to the proximal side 54 of the bronchial lumen 52 and a distal section 64 is positioned adjacent to the distal side 56 of the bronchial lumen 52. The body portion 50 is comprised of a tubular member 66 that has a radially collapsible cylindrical configuration. In an exemplary embodiment, the tubular member 66 has an axial length of about 10 millimeters (mm) to 30 mm although the length could also be outside this range. The tubular member has an external diameter that would permit the member to disposed within various bronchial passageways in the bronchial tree, or other intracorporeal lumens. The average diameter of a bronchial passageway is about 10 mm, although it should be appreciated that the diameter of a bronchial passageway can vary for a specific patient and the location in the bronchial tree. In one embodiment, the outer diameter of the tubular member is about 5 mm to about 15 mm, although the diameter can vary based on the size of the bronchial passageway. The tubular member 66 can be made of a compliant, nonporous material such as silicone, PTFE or the like, but can also be made from polyurethane.

The tubular member 66 is sealed along a portion of an external surface 68 to the internal surface 70 of the bronchial lumen 52 by a sealing member 72, which has a transverse dimension that matches the transverse dimension of the internal surface 70 of the bronchial lumen 52. In this regard, the sealing member 72 seals the device externally to the internal surface 70 of the bronchial lumen 52 and prevents the passage of fluid in either direction, around the device between the external wall of the device 50 and the bronchial lumen 52.

A self-expanding retainer 74 is disposed circumferentially within the tubular member 66 and about the chamber 60. The retainer 74 secures the placement of the device 50 within the bronchial lumen 52 by exerting an outward pressure against the body portion 58 of the pump device 50 and the bronchial lumen 52. The self-expanding retainer 74 is made of an expandable material, such as out of a laser cut Nitinol tube, but can alternately be made of materials such as stainless steel or the like or have various configurations such as a spring, a coil shape or the like.

The body portion 58 of the pump device 50 circumferentially encloses a chamber 60 and has a proximal one-way valve 76 disposed at the proximal section 62 of the body portion 58 and a distal one-way valve 78 disposed at the distal section 64 of the body portion 58. The proximal one-way valve 76 and the distal one-way valve 78 are positioned to cooperatively allow the unidirectional flow of fluid through the chamber 60. Changes in volume of the compliant tubular member 66 pump fluid through the device 50 and the bronchial lumen 52.

The device 50 is positioned within the bronchial lumen such that the valves allow the flow of fluid in a desired direction. For example, the device can cause fluid to flow from a distal section of the lung via the distal side 56 of the bronchial lumen 52, through the chamber 60, and out of the body via the proximal side 54 of the bronchial lumen 52. The valves can prevent the flow of fluid through the device 50 in the inhalation direction. The proximal one-way valve 76 and the distal one-way valve 78 are designed to open in response to pressure changes within the chamber 60, which can occur with volume changes within the chamber 60 (such that the valves in another embodiment do not typically open in response to normal expiratory pressures). In other embodiments one or both of the valves can be configured to open during normal lung expiratory pressures. The proximal one-way valve 76 and the distal one-way valve 78 have a duckbill configuration but can alternately have other configurations, such as, for example, a poppet, ball, duckbill, Heimlich, flap, diaphragm, and leaflet valve or alterations and combinations thereof.

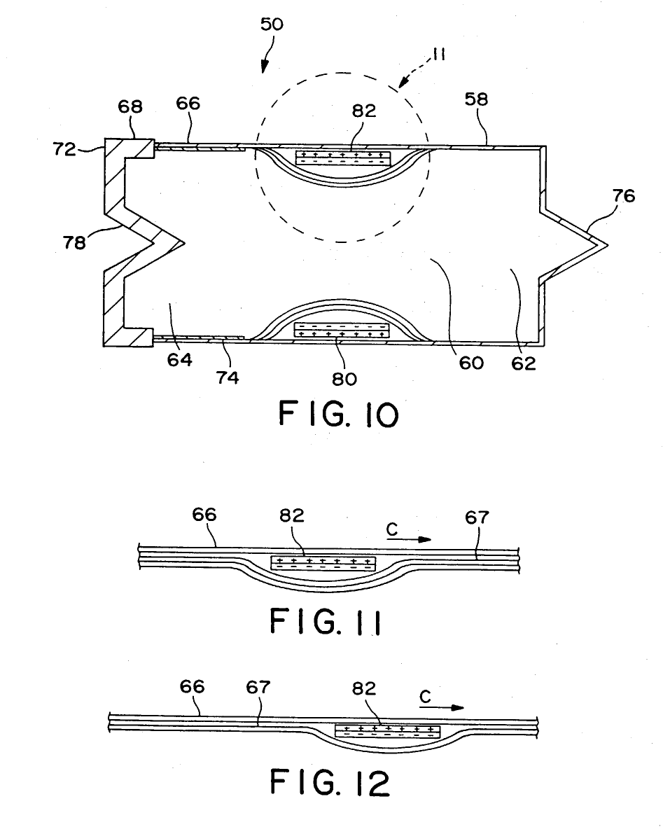

Disposed within the tubular member 66 at opposing positions are two magnetic elements that act as activation or actuation members to alter the chamber volume. That is, the magnets act as a drive mechanism that causes the chamber to change volume and propel fluid. The magnetic elements are comprised of a first magnet 80 and a second magnet 82. The magnets 80, 82 are made of a magnetic material, such as a rare earth magnet made of neodymium, but can also be made of other metals, alloys or the like. In other embodiments the magnetic elements can be made from ceramic materials and the like. The first magnet 80 and the second magnet 82 are disposed about the chamber 60 in opposing positions and are attached to the tubular member 66 and sealed within layers 67 of the tubular member 66 as shown in FIGS. 9 and 10. The magnetic elements can be secured to the tubular member 66 such that they have minimal or substantially no motion along a lateral axis or the magnetic elements can be disposed within the layers such that they can move along a lateral axis (line C) in a direction toward the proximal section 62 of the body portion 58 of the pump device 50, as shown in FIGS. 11 and 12, and move in a direction toward the distal section 64. The first magnet 80 and the second magnet 82 can also be embedded within a membrane and attached to the tubular member 66 or attached to the tubular member 66 and sealed by a membrane such that there are a variety of possible configurations and placements of the magnetic elements about the chamber.

The first magnet 80 and the second magnet 82 are oriented within the pump device 50 such that the polarity is in the same direction relative to the center of the chamber 60, which creates a repulsion of the magnets toward the center of the chamber 60. The tubular member 66 can be supported by the self-expanding retainer 74, which can comprise, for example, a Nitinol stent. In additional embodiments of the device the tubular member 66 can be supported by the repelling force of the magnets. For example, the first magnet 80 and the second magnet 82 each have the negative pole facing the center of the chamber 60. However, in additional embodiments the magnets can have either the positive pole facing the chamber 60 or have the negative pole toward the chamber 60.

The pump device 50 is placed within the bronchial lumen 52 to have an orientation such that the first magnet 80 is positioned relative to the portion of the bronchial lumen 52 that is most proximal (more external or superficial) to the patient's chest wall and the second magnet 82 is oriented more distal (more internal or deeper) to the chest wall.

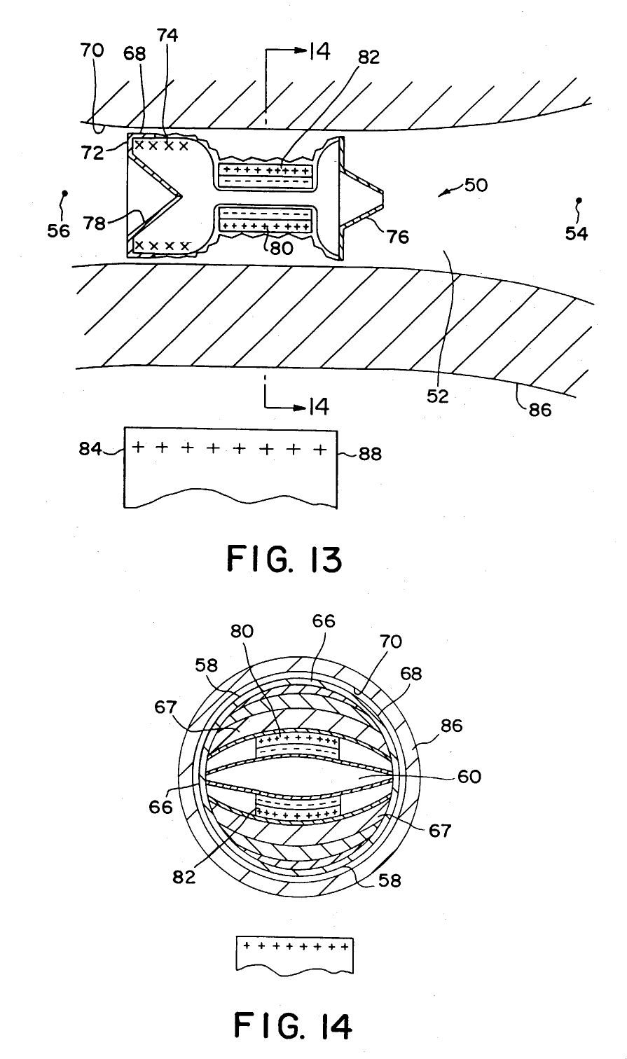

As shown in FIGS. 13 and 14, an external magnet 88 is placed near the chest wall 86, or an electromagnet is switched on near the chest wall 86, such that the patient is exposed to a pulsed magnetic or electromagnetic field from a single direction oriented perpendicular to the pump device 50. As shown in FIG. 13, when the external magnet 88 has a polarity that opposes the external charge of the proximal first magnet 80 (a positive charge as shown in FIG. 13), the distal second magnet 82 is drawn toward the chest wall 86, and also toward the center of the chamber 60, while the proximal first magnet 80 is repelled. This causes a reduction of the chamber volume and forces fluid through the proximal one-way valve 76. The external magnet 88 can be intermittently removed or replaced proximal to the chest wall 86, the polarity repeatedly reversed, or in the case of an electromagnet, switched on and off to effect a desired pumping action.

The first magnet 80 and the second magnet 82 can be designed and positioned within the device so that they pump effectively in a variety of bronchial shapes and function in a variety of positions and angles such as when the patient is supine, prone, sitting upright or standing. Additional embodiments can also include one or more magnetic elements disposed within the device.

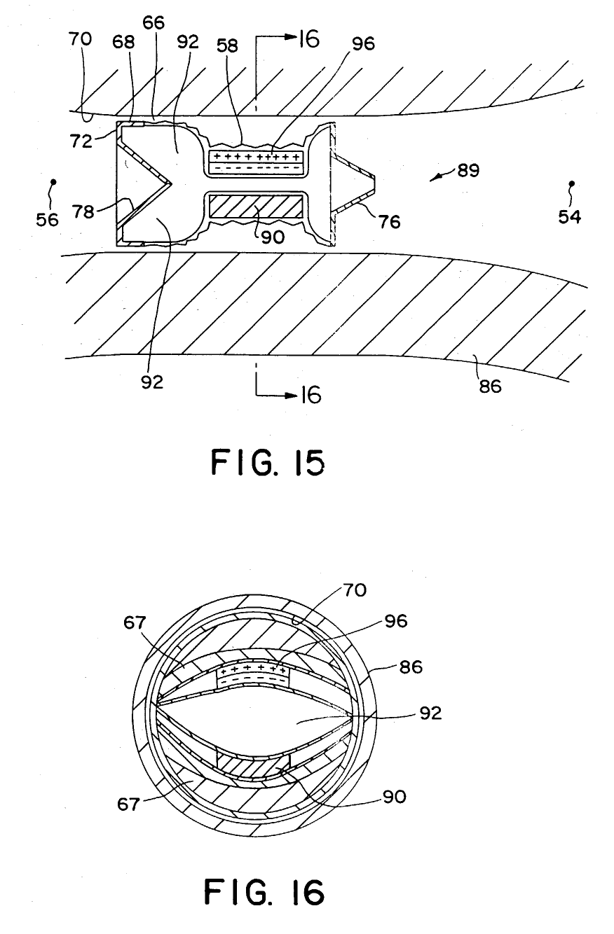

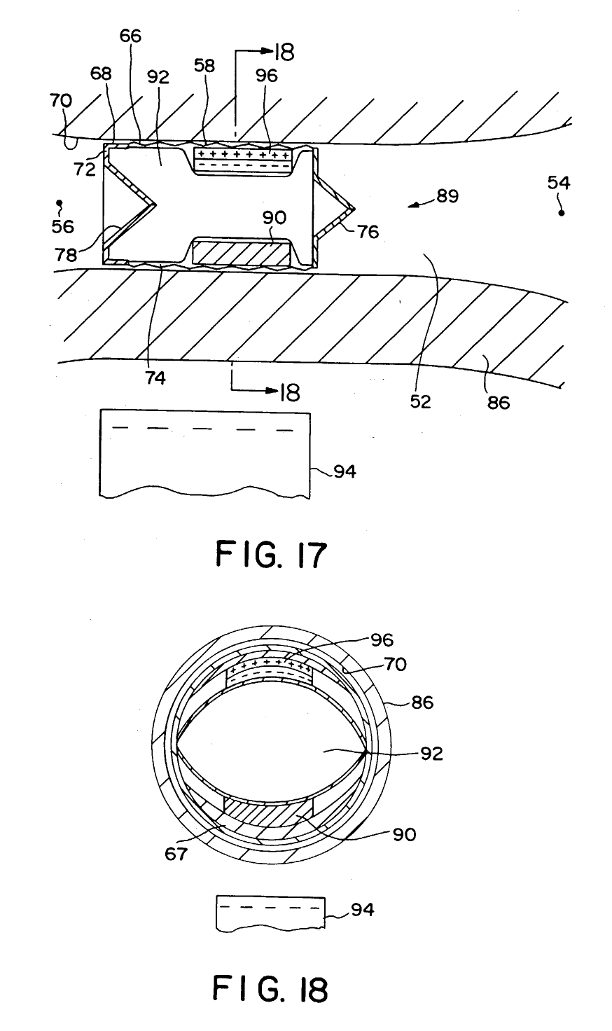

FIGS. 15-18 illustrate another embodiment of a magnetically driven active pump device 89, similar to the device shown in FIGS. 8-14, in which a ferrous metal plate 90 is used in place of the first magnet 80. The device 89 is positioned within the bronchial lumen 52 such that the ferrous metal plate 90 is positioned about the side of the chamber 92 of the device 89 that is most proximal to the chest wall 86 where the external magnet 94 is positioned. A second magnetic element comprised of a magnet 96 is disposed about the chamber 92 in a position that opposes the ferrous metal plate 90 and is more distal (more internal) to chest wall 86 than the ferrous metal plate 90. In the absence of an external magnet, the magnet 96 attracts the ferrous metal plate 90 and the chamber is contracted as shown in FIGS. 15 and 16. When the external magnet 94 having a negative charge is positioned proximal to the body, it attracts the ferrous metal plate 90 but repels the magnet 96 thereby driving the ferrous metal plate 90 and the magnet 96 apart and expanding or opening the pumping chamber as shown in FIGS. 17 and 18. Intermittently removing the external magnet from the proximity of the chest wall 86 and replacing it adjacent to the chest wall 86, rotating or reversing the polarity, or switching an electromagnet on or off can effect a pumping action.

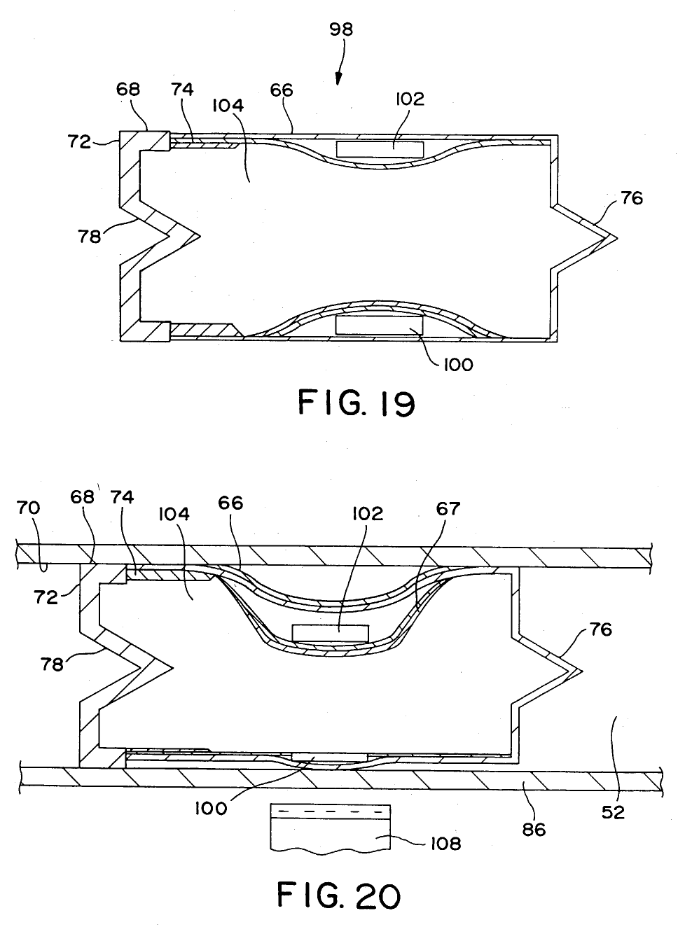

FIGS. 19-21 illustrate an alternate embodiment of a magnetically driven active pump device 98, similar to the pump device 50 shown in FIGS. 8-14, in which a first ferrous metal plate 100 and a second ferrous metal plate 102 are used in place of the first magnet 80 and the second magnet 82. The first ferrous metal plate 100 and the second ferrous metal plate 102 are disposed about opposing sides of a chamber 104. The ferrous metal plates are made of martenistic stainless steel, such as 17-4 PH or 400-series stainless steel such that they are resistant to corrosion, but can also be made of other materials with like properties. As described in previous embodiments the ferrous plates can be attached to or disposed within a layer of the tubular member 106. As shown in FIG. 16 the device can be positioned in a bronchial lumen 52 and an external magnetic element such as an external magnet 108 can be positioned adjacent to the chest wall 86 along an axis passing through the two ferrous metal plates such that the first ferrous metal plate 100 and the second ferrous metal plate 102 are drawn toward the chest wall 86. This compresses the pumping chamber 104 against the bronchial wall most proximal to the external magnet 108.

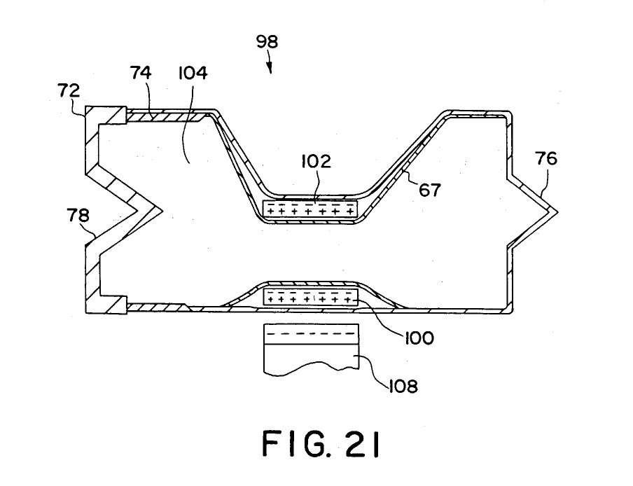

As a secondary effect, the magnet also induces a first charge on the sides of the first ferrous metal plate 100 and the side of the second ferrous metal plate 102 closest to the external magnet 108, the sides of the plates which face the external magnet 108. The magnet also induces a second charge opposite the first charge on the sides farther away from the external magnet 108. The first ferrous metal plate 100 and the second ferrous metal plate 102 would therefore be drawn closer together and further constrict the chamber due to attraction of the dissimilar charges internally toward the chamber. For example, the two sides of the first ferrous metal plate 100 and the second ferrous metal plate 102 that face each other would have a negative and a positive magnetic charge, respectively, and draw further draw the plates together as shown in FIG. 21.

A number of other embodiments having various geometries and arrangements of metal/and or magnetic elements and may be defined such that an external magnetic force is used to develop a driving force to pump fluid out of a lung segment. This concept can also be used in a variety of other intracorporeal lumens and/or positioned throughout the body.

Fixed Volume Chamber Pump Device

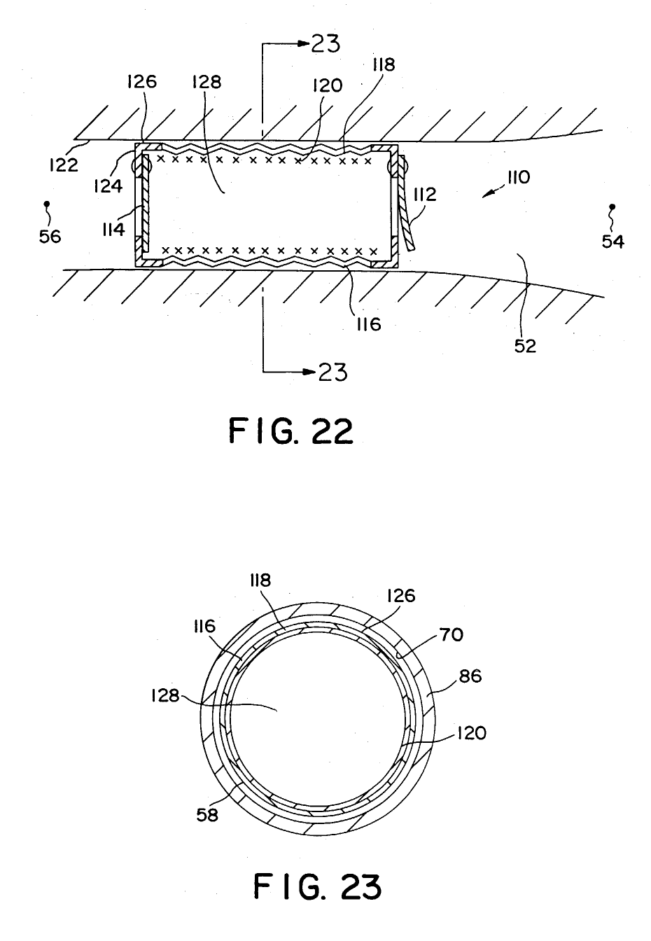

FIGS. 22 and 23 show another embodiment of the present device comprised of a fixed volume chamber pump device 110, which is shown positioned within a bronchial lumen 52. This device is typically used to expel fluid from a distal portion of the lung when the lung is “pressurized” such as when the intrathoracic pressure increases. The pump device 110 is generally activated when the pressure is varied between the proximal side 54 of the bronchial lumen 52 and the distal side 56 of the bronchial lumen 52. For example, “pressurization” of the lung can be achieved when straining to exhale against a closed mouth or glottis, performing a valsalva maneuver or coughing. Such actions can typically cause the pressure to increase throughout the entire lung, including an isolated or distal diseased lung segment.

These “pressurization” techniques can act to equilibrate pressures within the lung and the airway such that pressure in the proximal side 54 of the bronchial lumen is increased, the pressure within the fixed volume chamber device 110 is relatively unchanged and the pressure in the distal side 56 of the bronchial lumen 52 is increased. The resulting pressure differential can force fluid into the fixed volume chamber device 110 from the distal side 56 of the bronchial lumen 52. When the stimulus (pressurizing technique) is released there is a reduced pressure in the proximal airway (proximal side 54 of the bronchial lumen 52) and a substantially unchanged pressure within the fixed-volume device 110. This results in a flow of fluid out of the device 110 into the proximal side 54 of the bronchial lumen 52.

Therefore, when the entire lung is pressurized, fluid pressure will increase in a distal, isolated lung segment, but fluid pressure will not substantially increase within the chamber 128 between the two valves 112, 114. Therefore, fluid will be forced from a distal lung segment through the distal valve into the chamber. When the pressure is released, the fluid will flow through the proximal valve and out of the lung.

As shown in FIG. 22, the device 110 has a proximal one-way valve 112 and a distal one-way valve 114. As discussed above the device 110 is oriented within a bronchial lumen 52 such that the device 110 will pump fluid from the distal side 56 of the bronchial lumen 52 to the proximal side 54 of the bronchial lumen 52. The device has a body portion 116 which is formed of a tubular member 118, which can be made, for example, of PTFE or the like. Within the tubular member 118 is a self-expanding member 120, that can be formed, for example, of a laser cut Nitinol tube. The tubular member can also have a configuration such as a stent, coil, spring or the like and be made from such materials as stainless steel, or the like.

The self-expanding member 120 exerts an outward force laterally against the wall of the tubular member 118 and the internal wall 122 of the bronchial lumen 52 to secure the device 110 within the bronchial lumen 52. The tubular member 118 also has a sealing member 124 which seals an external surface 126 of the tubular member 118 to the internal wall 122 of the bronchial lumen 52 and prevents the passage of fluid around the device 110 within an intracorporeal lumen in either direction, such that fluid does not pass between the sealing member 124 and the tubular member 118 or the sealing member 124 and the bronchial lumen 52.

The body portion 116 which is further comprised of the tubular member 118 forms a substantially fixed volume chamber 128 disposed between the proximal one-way valve 112 and the distal one-way valve 114. The proximal one-way valve 112 and the distal one-way valve can be flap valves which are sealed to the body portion 116 and positioned to allow the uni-directional flow of fluid through the chamber 128. The proximal one-way valve 112 and the distal one-way valve 114 can also have various shapes such as, for example, poppet, diaphragm, leaflet, Heimlich, duckbill or various other valve configurations and combinations thereof. The volume of the chamber 128 is relatively constant and fluid is pumped through the chamber 128 in response to the pressure changes within the distal side 56 of the bronchial lumen 52 and the proximal side 54 of the bronchial lumen 52.

The use of the fixed volume chamber pump device 110 can express a desired volume of fluid per each pressurization episode (each performance of the pressurization technique or stimulus). Specifically, it is understood that a typical person can generate lung pressure of 2-4 pounds per square inch (psi) when coughing or straining to inflate a balloon. The emphysematic patient can typically generate half that pressure, or 1-2 psi, which is still a 7-14% increase in pressure over standard atmospheric pressure. Applying this pressure increase could drive fluid into the chamber 128 from the distal side 56 of the bronchial lumen 52. When the pressure is released, this fluid should flow out of the proximal one-way valve 112 and out of the lungs. The proximal one-way valve 112 and the distal one-way valve 114 have cracking pressures that are preferably sufficiently low so that the valves can expel as much fluid as possible. As this exercise is repeated, fluid is expressed out of the isolated lung region and through the pump.

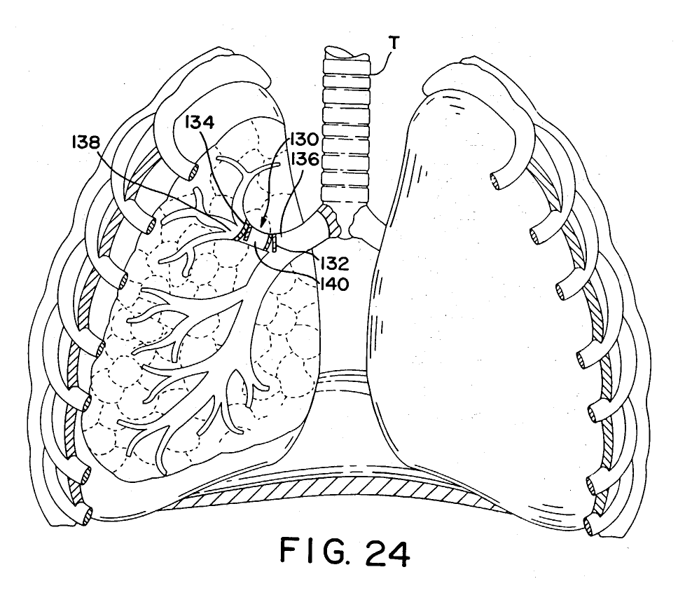

In other embodiments a fixed volume pump device 130 can include the placement of a one-way proximal valve 132 and a one-way distal valve 134 positioned to allow the unidirectional movement of fluid flow from a distal segment of the lung proximally toward the trachea and prevent fluid flow in the opposite direction. As shown in FIG. 24, this particular embodiment is generally comprised of two valves which may be placed in proximal bronchial branches such as off of the main upper lobe bronchus, lower lobe bronchus or right middle lobe bronchus, such as the segmental or sub-segmental bronchi. For example, placement of a first distal one-way valve 134 within a distal portion 138 of the main upper lobe bronchus 140 , or in other embodiments a branch off of the main upper lobe bronchus, and the positioning of a second proximal one-way valve 132 positioned at the proximal side 136 of the main upper lobe bronchus 140 could create a chamber having a volume of approximately 3 milliliters (ml). Because this portion of the main upper lobe bronchus 140 is relatively incompressible due to its partial composition of cartilaginous rings it could impart a relatively constant volume between the two valves. The main upper lobe bronchus 140 could be made more incompressible, and thus the volume could further be controlled or maintained, with the implantation of stents at a desired site within the main upper lobe bronchus 140 or within another desired site of the bronchus.

Pump Device with Movable Weight

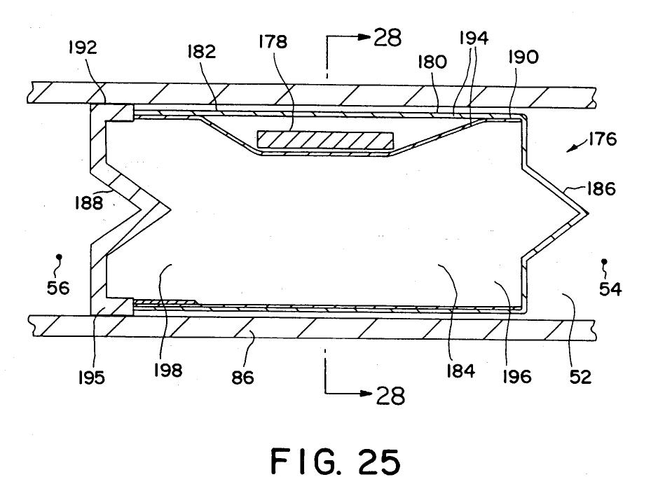

FIG. 25 shows an embodiment of an active pump device 176 having a movable weight 178. The pump device 176 is formed of a body portion 180 which is an elongate tubular member 182 which forms an internally disposed chamber 184. A proximal one-way valve 186 and a distal one-way valve 188 are disposed and sealed to the tubular member at proximal end 190 and distal end 192, respectively, to cooperatively allow for the unidirectional flow of fluid through the chamber 184. The tubular member can be made, for example, of PTFE or the like. The proximal one-way valve 186 and the distal one-way valve are shown as duckbill valves but can alternately have various configurations such as, for example, leaflet, poppet, Heimlich, reed, diaphragm, or combinations thereof. The movable weight 178 is disposed about the chamber 184 and typically sealed within one or more layers 194 of the tubular member so it is not exposed to fluid or mucous within the chamber. The moveable weight 178 is typically comprised of stainless steel, or the like. The direct action of the movable weight 178 is used to compress the chamber and effect a volume change within the chamber.

The pump device 176 can be placed in an intracorporeal lumen such as a bronchial lumen 52 and sealed to the lumen 52 with a sealing member 195, such that fluid flow does not pass around the device 176 in either direction. The device 176 is positioned to allow fluid flow from a distal side 56 of the bronchial lumen 52 to a proximal side of a bronchial lumen 54. When the movable weight 178 compresses the chamber 184, there is a volume change between a proximal portion 196 of the chamber 184 and the proximal side 54 of the bronchial lumen 52 that forces fluid out of the chamber 184, and a volume change between a distal portion 198 of the chamber 184 and the distal side 56 of the bronchial lumen 52 that draws fluid into the chamber 184. In other embodiments a movable weight could be disposed within the pump device 176 such that it is attached to a spring, which directly or indirectly activates the volume changes in the device or pump. The movement of the movable weight could wind the spring which can also have a ratchet to prevent “unwinding”. The movable weight 178 is designed to generate a maximum force and have the capacity to travel in any orientation such that it may be placed in a variety of positions within an intracorporeal lumen or pulmonary lumen and move in response to movements created by the patient during the activities of daily living or by the performance of specific exercises.

Counter-Pulsing Control of Pump Device

The pump device can be controlled in a desired pulsing cycle that works in cooperation with the patients breathing cycle. In such a case, the pump works counter to open air-ways during inspiration but does not work counter to airways during exhalation. Therefore, the methods described herein utilize in-situ pumping in a manner that takes advantage of the patient's open airways during inspiration while not working against the closed airways during exhalation.

The rapid frequency of the pulsing cycle of the pump allows for fluid to be drawn into the pump device from a distal portion of a bronchial lumen and hence draw fluid from a distal segment of the lung with such pulsed timing that maintains the patency of the airway throughout the activation cycle of the pump, which includes the peak of the suction wave prior to the next cycle. Thus, the cyclical quick pulsing of the pump during inspiration by the patient and followed by cessation of the pumping action during expiration of the patient allows for fluid to be removed from a distal lung segment. It also allows for the bronchial segment to remain open and thus maintain the patency of the bronchial lumen. The pump frequency can be set such that the pulsing cycle can provide several quick pulses, such as 1 pulse per second, although the rate can vary. The pump force is also regulated to generate a negative pressure which allows for fluid to be drawn into the pump from a distal segment of the lung while maintaining the patency of the distal airways. The cycle of the pump is set to activate the pump counter to the patient's respiratory cycle such that the negative pressure is applied during the inspiration phase by the patient, when the lung's tethering forces can act to keep the distal airways open and the fluid flow through the pump and out of the proximal side of the bronchial lumen is retrograde (in the expiratory direction).

The counter pulsing action of the pump device can be regulated automatically or manually by the patient. In embodiments which include the automated regulation of the pump device, a monitor is attached to the patient that measures the patient's respiratory cycle and activates the pump counter to that cycle, e.g. activates the pump to draw fluid into the device from a distal portion of the lung during the inhalation phase of the patient such that fluid is pumped in a direction counter (in an expiratory direction) to the air inhaled.

Manual regulation of the pump device in a counter-pulsing manner can include the determination of the patient's baseline breathing rate (the respiratory cycle of inspiration and exhalation) and setting the pump to cycle at the same frequency. The expiration phase of the pump device can be set to correspond with the time of the patients inspiratory wave and the rebound phase of the pump device can be timed to the length of the patient's normal expiratory wave. The device can further include a feedback apparatus or the use of a feedback mechanism, such as a flashing light, different colored lights, or a sound such as a bell or a beep with varying frequencies to signal the patient as to the phase of the pumping cycle. The patient can then self regulate their breathing cycle to breathe out (exhale) when the pump is in the rebound phase and the breath in (inhale) when the pump is in the activated to pump out fluid from the distal portion of the lung.

Alternate embodiments are directed toward a method of assisting expiration from a patient's lung. The method includes implanting a pump into a bronchial lumen that fluidly communicates with the lung and operating the pump so that the pump causes gas to flow out of the patient's lung through the bronchial lumen while the pump is positioned within the bronchial lumen.

Alternate embodiments can be directed toward a method of fluid removal from an intracorporeal lumen or a distal segment of the lung. The method includes advancing a pump device through a patients pulmonary system and the placement of the pump device within a bronchial lumen. The pump device can be any of the pump devices described herein. The pump device can be sealed the bronchial lumen with a sealing membrane which prevents the passage of fluid around the device. The pump can then be actuated to cause the flow of fluid through the chamber in and expiratory direction. The pump can include an actuation member comprised of a temperature-sensitive shape-memory alloy, magnets or magnetic elements, such as ferrous plates, or movable weights. The actuation member can then be effected by various intrinsic and extrinsic sources such that the temperature shape-memory alloy is activated by temperature changes which occur during normal or temperature controlled breathing of the patient. The placement of a magnet adjacent to the chest wall, or switching on and off of an electromagnet can effect a volume change in the chamber of a magnetically driven pump while everyday movement or specific exercise can effect the moving weight driven pump.

Method of Implanting the Pump Device

The pump device can be implanted into a bronchial lumen in a variety of manners, such as by using a delivery device that is inserted into the bronchial lumen through the trachea. Alternately, the pump device can be surgically inserted into the bronchial lumen. If a delivery catheter is used, the delivery catheter is inserted into the bronchial passageway so that the pump device is positioned at a desired location in the bronchial passageway. This can be accomplished by inserting a distal end of the delivery catheter into the patient's mouth or nose, through the trachea, and down to the target location in the bronchial passageway.

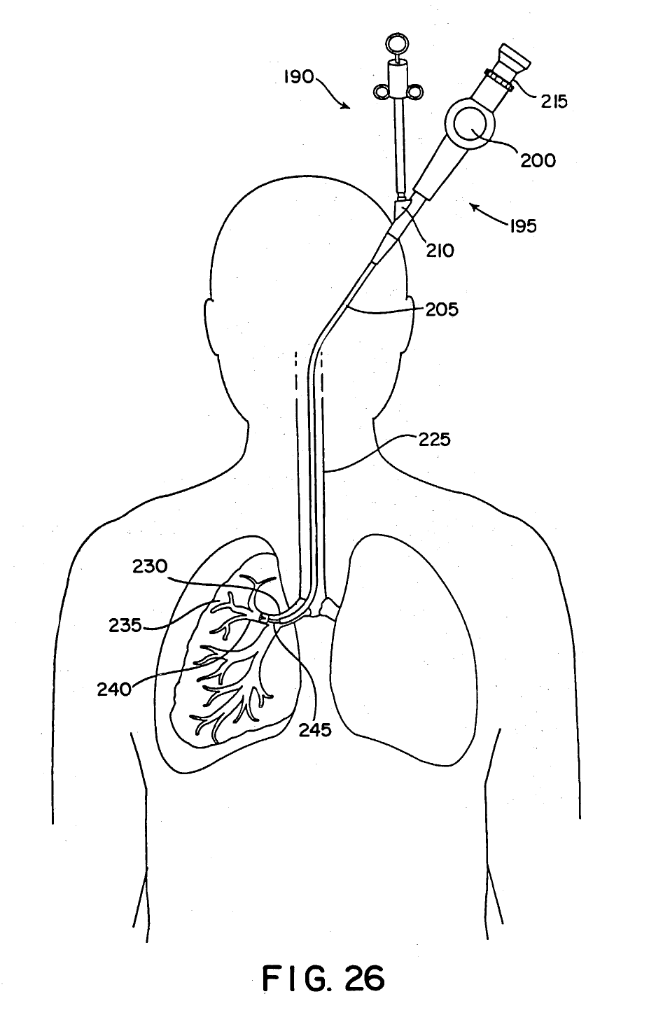

The delivery of the delivery catheter to the bronchial passageway can be accomplished in a variety of manners. In one embodiment, a bronchoscope is used to deliver the delivery catheter. For example, with reference to FIG. 26, a delivery catheter 190 can be deployed using a bronchoscope 195, which in an exemplary embodiment has a steering mechanism 200, a shaft 205, a working channel entry port 210, and a visualization eyepiece 215. The bronchoscope 195 has been passed into a patient's trachea 225 and guided into the right primary bronchus 235 of the patient according to well-known methods. It should be appreciated that, if a bronchoscope is used to deliver the pump device, the pump device should be sufficiently small to fit within the delivery channel of the bronchoscope or the delivery channel should be sufficiently large to receive the pump device.

In one embodiment, the distal end of the bronchoscope is deployed to a location that is at least one bronchial branch proximal to the target bronchial lumen where the pump device will be implanted. If the distal end of the bronchoscope is inserted into the target bronchial lumen, it can be difficult, if not impossible, to properly visualize and control the deployment of the pump device in the target bronchial lumen. For example, if the bronchoscope is advanced into the right primary bronchus 235 as shown in FIG. 26, the right upper lobar bronchi 240 can be visualized through the visualization eyepiece of the bronchoscope. The right upper lobar bronchi 240 is selected as the target location for placement of a pump device and the distal end of the bronchoscope is positioned one bronchial generation proximal of the bronchial passageway for the target location. Thus, the distal end of the bronchoscope is deployed in the right primary bronchus 235. The delivery catheter 190 is then deployed down a working channel (not shown) of the bronchoscope shaft 205. The distal end 245 of the catheter 190 is then guided out of the distal tip of the bronchoscope and advanced distally until the delivery system housing containing the compressed pump device is located inside the lobar bronchi 240.

Alternately, the delivery catheter 190 can be fed into the bronchoscope working channel prior to deploying the bronchoscope to the bronchial passageway. The delivery catheter 190 and the bronchoscope 195 can then both be delivered to the bronchial passageway to the target passageway as a single unit. The delivery catheter can then be advanced into the target bronchi as before, and the pump device 110 delivered.

In another embodiment, the catheter 190 is deployed using a guidewire that guides the catheter 190 to the delivery site. In this regard, the delivery catheter 190 could have a well-known steering function, which would allow the catheter 190 to be delivered with or without use of a guidewire.

Visualization of the progress of the distal tip of the delivery catheter 190 can be provided by a bronchoscope that is manually advanced in parallel and behind the delivery catheter 190. Visualization or imaging can also be provided by a fiber optic bundle that is inside the delivery catheter 190.

Although embodiments of the present device and methods of use thereof are described in detail with reference to certain versions, other versions, embodiments, methods of use, and combinations thereof are also possible. Therefore the spirit and scope of the appended claims should not be limited to the description of the embodiments contained herein.

What is claimed is:

1. An implantable pump for assisting expiration of fluid from a lung, comprising:
a housing defining a chamber, the housing dimensioned for implantation in a bronchial lumen;
a fluid propulsion mechanism attached to the housing in fluid communication with the chamber, the fluid propulsion mechanism positioned to propel fluid through the chamber;
a retainer coupled to the housing and configured to engage a wall of the bronchial lumen to maintain the pump in a fixed position within the bronchial lumen.

2. The pump of claim 1, wherein the propulsion mechanism comprises a fan.

3. The pump of claim 1, wherein the propulsion mechanism alters the volume of the chamber to force fluid into and out of the chamber.

4. The pump of claim 3, wherein the propulsion mechanism comprises a bellows that expands and contracts to alter the volume of the chamber.

5. The pump of claim 3, wherein the propulsion mechanism comprises a plunger movably mounted in the chamber, wherein the plunger moves within the chamber to vary the volume of the chamber.

6. The pump of claim 3, wherein the propulsion mechanism comprises a temperature sensitive material that changes shape to alter the volume of the chamber in response to a change in temperature.

7. The pump of claim 1, further comprising a first one-way valve attached to the housing and in fluid communication with the chamber.

8. The pump of claim 7, further comprising a second one-way valve attached to the housing and in fluid communication with the chamber.

9. The pump of claim 1, further comprising a drive mechanism coupled to the propulsion mechanism, wherein the drive mechanism interacts with the propulsion mechanism to cause the propulsion mechanism to propel fluid through the chamber.

10. The pump of claim 9, wherein the drive mechanism includes a magnet that can be exposed to a magnetic force to cause the propulsion mechanism to propel fluid through the chamber.

11. The pump of claim 9, wherein the drive mechanism includes a shape-memory material.

12. The pump of claim 9, wherein the drive mechanism utilizes a change in temperature to cause the propulsion mechanism to propel fluid through the chamber.

13. The pump of claim 1, further comprising a power supply coupled to the propulsion mechanism to provide power to the propulsion mechanism.

14. The pump of claim 13, wherein the power supply comprises an electric battery.

15. The pump of claim 13, wherein the power supply comprises a patient's muscular system.

16. The pump of claim 13, wherein the power supply comprises gravity.

17. The pump of claim 1, wherein the pump can be reduced in size to facilitate insertion of the pump into the bronchial lumen.

18. The pump of claim 1, wherein the retainer comprises a stent.

19. The pump of claim 18, wherein the stent is self-expanding.

20. The pump of claim 1, additionally comprising a seal attached to the housing, wherein the seal has a surface that can seal to an interior wall of the bronchial lumen.

21. A pump device for intracorporeal placement in an intracorporeal lumen of a patient, comprising:
a body portion having proximal and distal sections, the body portion forming a chamber;
an outside sealing member attached to the body portion, the sealing member including an outside sealing surface configured to seal to the intracorporeal lumen and separate the intracorporeal lumen into proximal and distal volumes;
a first one-way valve member attached to the body portion and in fluid communication with the chamber and configured to allow fluid flow out of the chamber, and into the proximal volume of the intracorporeal lumen;
a second one-way valve member attached to the body portion and in fluid communication with the chamber and configured to allow fluid flow into the chamber from the distal volume of the intracorporeal lumen, wherein the first and second one-way valve members cooperatively allow unidirectional flow through the chamber; and
an actuation member coupled to the chamber and configured to alter the chamber volume to effect a pumping action from the distal volume of the intracorporeal lumen to the proximal volume of the intracorporeal lumen.

22. The pump device of claim 21, wherein the body portion comprises a tubular member.

23. The pump device of claim 21, wherein the actuation member is a temperature responsive shape-memory material, a magnetically responsive material or one or more movable weights.

24. The pump device of claim 21, wherein the actuation member is intrinsically driven.

25. The pump device of claim 21, wherein the actuation member is extrinsically driven.

26. The pump device of claim 21, wherein the body portion includes a bellows disposed about the chamber, wherein the bellows can expand and contract between an expanded state wherein the chamber has a first volume, and a contracted state wherein the chamber has a second volume that is smaller than the first volume.

27. The pump device of claim 21, wherein the actuation member is a temperature-sensitive shape-memory member disposed circumferentially about the chamber and responsive to temperature changes within the lumen.

28. The pump device of claim 21, wherein the actuation member can constrict to effect the alteration of the chamber volume which effects a pressure change between the chamber and the proximal volume of the lumen.

29. The pump device of claim 21, wherein the actuation member can constrict to effect the alteration of the chamber volume which effects a pressure change between the chamber and the distal volume of the lumen.

30. The pump device of claim 21, wherein the actuation member can expand to effect the alteration of the chamber volume which effects a pressure change between the chamber and the distal volume of the lumen.

31. The pump device of claim 21, wherein the actuation member is responsive to the mechanics of the patient's respiratory cycle.

32. The pump device of claim 21, wherein the first and second valve members are selected from the group consisting of poppet, ball, duckbill, Heimlich, flap, diaphragm, and leaflet.

33. The pump device of claim 21, wherein the actuation member is comprised of one or more magnets disposed about the chamber.

34. The pump device of claim 21, wherein the actuation member is further comprised of one or more ferrous metal elements disposed about the chamber.

35. The pump device of claim 21, wherein the actuation member is comprised of one or more magnets disposed about the chamber and one or more ferrous elements disposed about the chamber.

36. The pump device of claim 33, wherein the magnets are positioned to compress or expand the chamber in response to an extrinsic magnetic force.

37. The pump device of claim 34, wherein the ferrous elements are positioned to compress or expand the chamber in response to an extrinsic magnetic force.

38. The pump device of claim 35, wherein the one or more magnets and the one or more ferrous metal elements are positioned to compress or expand the chamber in response to an extrinsic magnetic force.

39. The pump device of claim 33, wherein the magnets are positioned such that they are of similar polarity internally relative to a longitudinal axis of the chamber.

40. The pump device of claim 34, wherein one or more ferrous elements are placed in opposing positions relative to a longitudinal axis of the chamber.

41. The pump device of claim 35, wherein the one or more ferrous elements is positioned opposite the one or more magnets and proximal to a desired site for the application of the extrinsic magnetic force.

42. The pump device of claim 21, wherein the actuation member is a movable weight disposed about the chamber that intermittently compresses the chamber.

43. The pump device of claim 42, wherein the weight is moved in response to respirations of the patient.

44. An implantable pump for assisting expiration of fluid from a lung, comprising:
a housing dimensioned for implantation in a bronchial lumen;
means for pumping fluid through the bronchial lumen, the means for pumping attached to the housing;
a retainer coupled to the housing and configured to engage a wall of the bronchial lumen to maintain the pump in a fixed position within the bronchial lumen.

45. The pump of claim 44, wherein the means for pumping comprises an impeller.

46. A method of assisting expiration from a patient's lung, comprising:
implanting a pump into a bronchial lumen that fluidly communicates with the lung;
operating the pump so that the pump causes gas to flow out of the patient's lung through the bronchial lumen while the pump is positioned within the bronchial lumen.

47. The method of claim 46, wherein operating the pump comprises applying a magnetic force to the pump so that the magnetic force moves a magnet of the pump so as to change an internal volume of a pump chamber to effect a pumping force that propels fluid through the pump.

48. The method of claim 46, wherein operating the pump comprises causing a bellows of the pump to expand and contract so as to change an internal volume of a pump chamber to effect a pumping force that propels fluid through the pump.

49. The method of claim 46, wherein operating the pump comprises applying a temperature change to the pump to cause a temperature-sensitive material of the pump to change shape so as to change an internal volume of a pump chamber to effect a pumping force that propels fluid through the pump.

50. The method of claim 46, wherein operating the pump comprises causing a fan of the pump to rotate and propel fluid through the pump.

51. The method of claim 46, wherein operating the pump comprises causing a plunger to move within an internal chamber of the pump to effect a pumping force that propels fluid through the pump.

52. The method of claim 46, wherein the pump is implanted using a delivery device that is inserted into the bronchial lumen through a trachea attached to the lumen.

53. The method of claim 46, wherein the delivery device is inserted through a working channel of a bronchoscope.

54. The method of claim 46, wherein the pump causes gas to flow out of the patient's lung in synchronization with the patient's breathing.

55. The method of claim 54, additionally comprising sensing movement of the patient's chest wall in order to synchronize with the patient's breathing.

56. The method of claim 54, additionally comprising sensing the patient's nerve impulses in order to synchronize with the patient's breathing.

57. The method of claim 54, additionally comprising sensing the a gas concentration of the lung in order to synchronize with the patient's breathing.

58. A method for the removal of fluid within an intracorporeal lumen or lung segment, the method comprising:
providing an intracorporeal pump device;
advancing the intracorporeal pump through a patient's pulmonary system;
placing the pump device within a bronchial lumen such that the pump device seals within the bronchial lumen; and
actuating the device to effect a unidirectional movement of fluid flow through the device in an expiratory direction from an internal segment of the lung.

59. The method of claim 58, wherein actuating the device is accomplished by producing a volume change in the chamber that allows the unidirectional movement of fluid flow through the chamber in an expiratory direction from an internal segment of the lung.

60. The method of claim 58, additionally comprising effecting a temperature change in the actuation member by heating or cooling the actuation member with air breathed by the patient.

61. The method of claim 60, additionally comprising placing an external magnetic source proximal to a chest wall of the patient to actuate the actuation member.

62. The method of claim 61, additionally comprising removing and replacing the external magnetic source in a cyclic manner.

63. The method of claim 61, additionally comprising reversing the polarity of the external magnetic source in a cyclic manner.

64. The method of claim 61, wherein the magnetic source is an electromagnet and wherein the method further comprises switching the electromagnet power source in a cyclic manner.

65. The method of claim 58, wherein the pump device comprises:
a body portion which forms a chamber and which has an outside sealing surface configured to seal the intracorporeal lumen;
a first valve member sealed to the body portion in fluid communication with the chamber configured to allow fluid flow out of but not into the chamber;
a second valve member sealed to the body portion, the sealing surface configured to allow fluid flow into but not out of the chamber, wherein the valves are positioned to cooperatively allow fluid flow through the chamber in a unilateral direction.

66. A method as defined in claim 65, additionally comprising:
increasing pressure in the lung to substantially increase pressure in both a proximal lumen portion and a distal lung segment to force air into the chamber; and
removing the pressure increase to allow fluid to flow from the chamber through the proximal valve.
